SEQUENCE LISTING (34 sequences)

SEQ ID NO: 1 - protein, 42 aa, Homo sapiens [sequence omitted]

SEQ ID NO: 2 - protein, 42 aa, Homo sapiens
DAEFRHDSGYEVHHQKLVSFAEDVGSNKGAIIGLMVGGVVIA

SEQ ID NO: 3 - protein, 42 aa, Homo sapiens
DAEFRHDSGYEVHHQKLVFFAEDVGSNKGAIIGLMVDGVVIA

SEQ ID NO: 4 - protein, 42 aa, Homo sapiens
DAEFRHDSGYEVHHQKLVSFAEDVGSNKGAIIGLMVDGVVIA

SEQ ID NO: 5 - DNA, 126 bp, Homo sapiens
gatgcggaatttcgccatgatagcggctatgaagtgcatcatcagaaactggtgagctttgcggaagatgtgggcagcaacaaaggcgcgattattggcctgatggtgggcggcgtggtgattgcg

SEQ ID NO: 6 - DNA, 126 bp, Homo sapiens; identical to SEQ ID NO: 5

SEQ ID NO: 7 - DNA, 126 bp, Homo sapiens
gatgcggaatttcgccatgatagcggctatgaagtgcatcatcagaaactggtgagctttgcggaagatgtgggcagcaacaaaggcgcgattattggcctgatggtggatggcgtggtgattgcg

SEQ ID NO: 8 - protein, 6 aa, Homo sapiens
KLVPFA

SEQ ID NO: 9 - protein, 6 aa, Homo sapiens
KLVDFA

SEQ ID NO: 10 - protein, 6 aa, Homo sapiens
KLVRFA

SEQ ID NO: 11 - protein, 6 aa, Homo sapiens
KLVCFA

SEQ ID NO: 12 - protein, 6 aa, Homo sapiens
KLVQFA

SEQ ID NO: 13 - protein, 6 aa, Homo sapiens
KLVGFA

SEQ ID NO: 14 - protein, 6 aa, Homo sapiens
KLVHFA

SEQ ID NO: 15 - protein, 6 aa, Homo sapiens
KLVKFA

SEQ ID NO: 16 - protein, 6 aa, Homo sapiens
KLVMFA

SEQ ID NO: 17 - protein, 6 aa, Homo sapiens
KLVSFA

SEQ ID NO: 18 - protein, 6 aa, Homo sapiens
RGVVIA

SEQ ID NO: 19 - protein, 6 aa, Homo sapiens
DGVVIA

SEQ ID NO: 20 - protein, 6 aa, Homo sapiens
CGVVIA

SEQ ID NO: 21 - protein, 6 aa, Homo sapiens
EGVVIA

SEQ ID NO: 22 - protein, 6 aa, Homo sapiens
QGVVIA

SEQ ID NO: 23 - protein, 6 aa, Homo sapiens
HGVVIA

SEQ ID NO: 24 - protein, 6 aa, Homo sapiens
IGVVIA

SEQ ID NO: 25 - protein, 6 aa, Homo sapiens
LGVVIA

SEQ ID NO: 26 - protein, 6 aa, Homo sapiens
KGVVIA

SEQ ID NO: 27 - protein, 6 aa, Homo sapiens
MGVVIA

SEQ ID NO: 28 - protein, 6 aa, Homo sapiens
FGVVIA

SEQ ID NO: 29 - protein, 6 aa, Homo sapiens
PGVVIA

SEQ ID NO: 30 - protein, 6 aa, Homo sapiens
SGVVIA

SEQ ID NO: 31 - protein, 6 aa, Homo sapiens
TGVVIA

SEQ ID NO: 32 - protein, 6 aa, Homo sapiens
WGVVIA

SEQ ID NO: 33 - protein, 6 aa, Homo sapiens
YGVVIA

SEQ ID NO: 34 - protein, 6 aa, Homo sapiens
VGVVIA

(19) United States
(12) Patent Application Publication
(10) Pub. No.: US 2017/0299614 A1
(43) Pub. Date: Oct. 19, 2017

(54) Alzheimer's Disease

(71) Applicant: The University of Sussex, Brighton (GB)

(72) Inventors: Louise Charlotte Serpell, Brighton (GB); Liza Dahal, Chelmsford (GB)

(21) Appl. No.: 15/501,811

(22) PCT Filed: Aug. 3, 2015
(86) PCT No.: PCT/GB2015/052242

§ 371 Date: Feb. 3, 2017

(30) Foreign Application Priority Data
Aug. 7, 2014 (GB) 1414038.8

(51) Int. Cl. G01N 33/68; C07K 14/47; G01N 33/96

(52) U.S. Cl. CPC G01N 33/6896; G01N 33/96; C07K 14/4711; G01N 2800/2821; G01N 2333/4709

(57) ABSTRACT

Variant forms of Amyloid-beta (Aβ) and kits comprising a variant Aβ are disclosed. The invention also provides uses of these kits and the Aβ variants in Aβ studies, for example in assays and methods for screening novel compounds for use in treating Alzheimer's Disease.

The invention relates to Alzheimer's disease (AD), and particularly, although not exclusively, to Amyloid-beta (Aβ or Abeta), and variants thereof. The invention also includes kits comprising a variant Aβ, and to uses of these kits and Aβ variants in Aβ studies, for example in assays and methods for screening novel compounds for use in treating AD.

Alzheimer's disease (AD) is characterized by the deposition of Aβ in extracellular amyloid plaques, as well as the intracellular accumulation of tau in neurofibrillary tangles in the brain. Mutations in Aβ and the Amyloid precursor protein (APP) are linked to familial AD, and therefore Aβ is thought to play an important role in the disease process. Numerous studies have been conducted to try to better understand how Aβ is involved in the neurodegeneration observed in AD patients and the symptoms of the disease. Some of these studies have been conducted in transgenic animals whilst others have used biomimetic membranes, cultured neurons or animal injection to investigate the direct effect of oligomeric and fibrillar Aβ in these systems.

Aβ is toxic and has been shown to cause membrane defects, neuronal cell death and effects on function (LTP) and to lead to changes in animal behaviour and neuronal networks. The Aβ peptide is a member of a larger group of amyloidogenic peptides and proteins (1) and it is believed that the toxic effect of these amyloidogenic peptides is linked to their ability to self-assemble to form β-sheet rich oligomeric species and cross-β structured amyloid fibrils.

Importantly, experimental controls in Aβ studies are limited to the use of a vehicle only (i.e. buffer), scrambled Aβ peptide, reverse Aβ, and two truncated forms of wild type Aβ, namely Aβ40 and Aβ1-28. Whilst these peptide controls provide some means for experimental control in Aβ studies, a problem with them is that they may also assemble to form oligomeric species or fibrils, which are poorly characterized. Also, whilst some of them share overall composition similarity with wild type Aβ, they differ significantly in sequence. Accordingly, the Aβ control peptides that are currently available are unsatisfactory in Aβ studies.

There is therefore a need for alternative Aβ peptides, which can act as suitable negatives control for use in Aβ studies, for example in test kits.

As described in the Examples, the inventors have designed a non-toxic variant of Aβ, which is non-aggregation prone, and which has been well-characterized for assembly, structure and toxic effect to provide a suitable control peptide for Aβ studies.

Hence, according to a first aspect, there is provided a variant Amyloid-beta (Aβ) peptide comprising a modified amino acid sequence of a wild-type Aβ peptide, wherein the modified amino acid peptide exhibits reduced propensity to aggregate compared to the wild type peptide.

Surprisingly, the inventors have managed to produce the variant form of Aβ according to the first aspect with at least one amino acid change that surprisingly transforms the wild type peptide from a strongly aggregating peptide with cytotoxic properties to a peptide that is unable to assemble or to cause toxicity in cell assays. The variant Aβ peptide is similar to the toxic wild type form of Aβ in terms of its sequence, PI, molecular weight, as well as other chemical characteristics, but, importantly, has been modified such that it does not aggregate to form β-sheet structures. Advantageously, the variant Aβ peptide of the invention can be used as a robust negative control for validating any effects of Aβ, for example in a test kit or for conducting an assay for screening novel compounds for use in treating AD. In addition, the variant Aβ peptide enables robust experiments to be performed for testing the efficacy of potential therapies that target Aβ toxicity or aggregation.

The amino acid sequence of wild-type Aβ(1-42) is known, and may be represented herein as SEQ ID No: 1, as follows:—

[SEQ ID No: 1]
DAEFRHDSGYEVHHQKLVFFAEDVGSNKGAIIGLMVGGVVIA

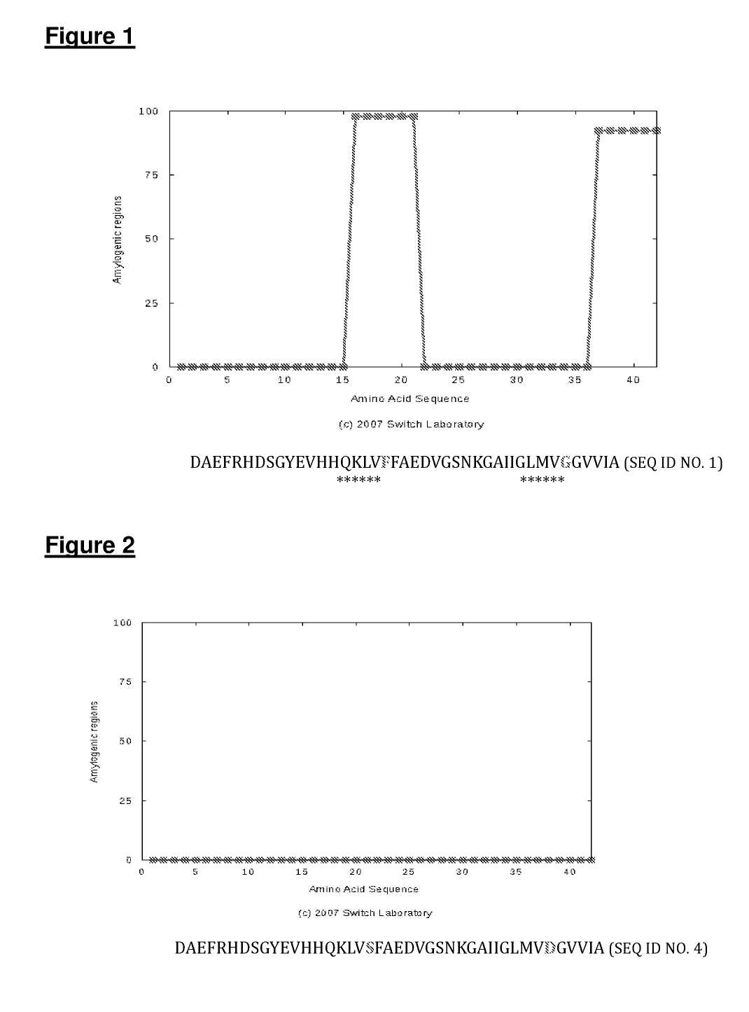

Preferably, therefore, the variant Amyloid-beta (Aβ) peptide of the first aspect comprises a modified amino acid sequence derived from the wild type Aβ peptide, which comprises an amino acid sequence substantially as set out in SEQ ID No: 1. FIG. 1 is a graph produced using WALTZ, and shows two peaks that indicate two amyloidogenic regions in the wild type Aβ(1-42) peptide (i.e. residues 16-21 and residues 37-42 of SEQ ID No: 1, which are shown bold and underlined above).

Thus, preferably the variant Amyloid-beta (Aβ) peptide of the invention comprises one or more modification in amino acids 16-21 or 37-42 of SEQ ID No: 1. Preferably, the variant Amyloid-beta (Aβ) peptide comprises at least two modifications in amino acids 16-21 or 37-42 of SEQ ID No: 1. Preferably, the variant Amyloid-beta (Aβ) peptide comprises at least one modification in amino acids 16-21 of SEQ ID No: 1 and at least one modification in amino acids 37-42 of SEQ ID No: 1. The or each modification is preferably a substitution.

The inventors tested numerous amino acid substitutions which were introduced into the two amyloidogenic regions in the wild type Aβ(1-42), and the amino acid substitutions that reduced the peaks for amyloidogenic regions were then shortlisted and summarised in Table 1. Based on their findings, the inventors were surprised to observe that residues 19 and 37 of SEQ ID No: 1 are particularly important for controlling Aβ toxicity and aggregation. Therefore, a preferred embodiment of the variant Amyloid-beta (Aβ) peptide is formed by modification of amino acid residue F19 or G37 of SEQ ID No: 1. Most preferably, the variant Amyloid-beta (Aβ) peptide is formed by modification of amino acid residue F19 and G37 of SEQ ID No: 1.

Preferably, the modification at amino acid residue F19 comprises a substitution with a serine. Hence, in one preferred embodiment, the variant Amyloid-beta (Aβ) peptide comprises an amino acid sequence substantially as set out in SEQ ID No: 2, as follows:—

[SEQ ID No: 2]
DAEFRHDSGYEVHHQKLVSFAEDVGSNKGAIIGLMVGGVVIA

Preferably, the modification at amino acid residue G37 comprises a substitution with an aspartic acid. Hence, in another preferred embodiment, the variant Amyloid-beta (Aβ) peptide comprises an amino acid sequence substantially as set out in SEQ ID No: 3, as follows:—

[SEQ ID No: 3]
DAEFRHDSGYEVHHQKLVFFAEDVGSNKGAIIGLMVDGVVIA

Preferably, the variant Amyloid-beta (Aβ) peptide comprises a F195 substitution or a G37D substitution. A most preferred variant Amyloid-beta (Aβ) peptide however comprises an F19S substitution and a G37D substitution. Hence, preferably the variant Amyloid-beta (Aβ) peptide comprises an amino acid sequence substantially as set out in SEQ ID No: 4, as follows:—

[SEQ ID No: 4]
DAEFRHDSGYEVHHQKLVSFAEDVGSNKGAIIGLMVDGVVIA

Hence, preferably the variant Amyloid-beta (Aβ) peptide comprises a double mutant based on the wild type sequence of SEQ ID No. 1.

The present invention also provides nucleic acids encoding embodiments of the variant Amyloid-beta (Aβ) peptide as defined herein.

Thus, in a second aspect, there is provided an isolated nucleic acid molecule encoding the variant Amyloid-beta (Aβ) peptide according to the first aspect.

Preferred nucleic acid molecules according to the second aspect of the invention may include:—
(a) Amyloid-beta (Aβ) with F19S substitution

[SEQ ID No: 5]
GATGCGGAATTTCGCCATGATAGCGGCTATGAAGTGCATCATCAGAAAC
TGGTGAGCTTTGCGGAAGATGTGGGCAGCAACAAAGGCGCGATTATTGG
CCTGATGGTGGGCGGCGTGGTGATTGCG

(b) Amyloid-beta (Aβ) with G37D substitution

[SEQ ID No: 6]
GATGCGGAATTTCGCCATGATAGCGGCTATGAAGTGCATCATCAGAAAC
TGGTGAGCTTTGCGGAAGATGTGGGCAGCAACAAAGGCGCGATTATTGG
CCTGATGGTGGGCGGCGTGGTGATTGCG

(c) Amyloid-beta (Aβ) with F19S & G37D substitution

[SEQ ID No: 7]
GATGCGGAATTTCGCCATGATAGCGGCTATGAAGTGCATCATCAGAAAC
TGGTGAGCTTTGCGGAAGATGTGGGCAGCAACAAAGGCGCGATTATTGG
CCTGATGGTGGATGGCGTGGTGATTGCG

The above nucleic acid sequences were produced using Backtranseq (www.ebi.ac.uk) using E. coli K12 as host. Preferably, therefore, the isolated nucleic acid molecule of the second aspect comprises a nucleotide sequence substantially as set out in any one of SEQ ID No: 5-7, or functional variant thereof. Most preferably, the nucleic acid molecule comprises a nucleotide sequence substantially as set out in SEQ ID No: 7, or functional variant thereof, i.e. encoding the double mutant.

The nucleic acid molecule may be an isolated or purified nucleic acid sequence. The nucleic acid sequence may be a DNA sequence. The nucleic acid molecule may comprise synthetic DNA. The nucleic acid molecule may comprise cDNA. The nucleic acid may be operably linked to a heterologous promoter. The nucleic acid sequence may be incorporated into a genetic construct for cloning purposes.

In a third aspect, therefore, there is provided a genetic construct comprising the nucleic acid molecule of the second aspect.

Genetic constructs of the invention may be in the form of an expression cassette, which may be suitable for expression of the encoded variant peptide in a host cell. The genetic construct may be introduced into a host cell without it being incorporated in a vector. For instance, the genetic construct, which may be a nucleic acid molecule, may be incorporated within a liposome or a virus particle. Alternatively, a purified nucleic acid molecule (e.g. histone-free DNA, or naked DNA) may be inserted directly into a host cell by suitable means, e.g. direct endocytotic uptake. The genetic construct may be introduced directly into cells of a host subject (e.g. a bacterial, eukaryotic or animal cell) by transfection, infection, electroporation, microinjection, cell fusion, protoplast fusion or ballistic bombardment. Alternatively, genetic constructs of the invention may be introduced directly into a host cell using a particle gun. Alternatively, the genetic construct may be harboured within a recombinant vector, for expression in a suitable host cell.

Therefore, in a fourth aspect, there is provided a recombinant vector comprising the genetic construct according to the third aspect.

The recombinant vector may be a plasmid, cosmid or phage. Such recombinant vectors are useful for transforming host cells with the genetic construct of the third aspect, and for replicating the expression cassette therein. The skilled technician will appreciate that genetic constructs of the invention may be combined with many types of backbone vector for expression purposes. Recombinant vectors may include a variety of other functional elements including a suitable promoter to initiate gene expression. For instance, the recombinant vector may be designed such that it autonomously replicates in the cytosol of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged.

The recombinant vector may also comprise DNA coding for a gene that may be used as a selectable marker in the cloning process, i.e. to enable selection of cells that have been transfected or transformed, and to enable the selection of cells harbouring vectors incorporating heterologous DNA. Alternatively, the selectable marker gene may be in a different vector to be used simultaneously with vector containing the gene of interest. The vector may also comprise DNA involved with regulating expression of the coding sequence, or for targeting the expressed polypeptide to a certain part of the host cell.

In a fifth aspect, there is provided a host cell comprising the genetic construct according to the third aspect, or the recombinant vector according to the fourth aspect.

The host cell may be a bacterial cell. The host cell may be an animal cell. The host cell may be a mammalian cell, for example a mouse or rat cell. It is preferred that the host cell is not a human cell. The host cell may be transformed with genetic constructs or vectors according to the invention, using known techniques. Suitable means for introducing the genetic construct into the host cell will depend on the type of cell.

In a sixth aspect, there is provided a transgenic host organism comprising at least one host cell according to the fifth aspect.

The genome of the host cell or the transgenic host organism of the invention may comprise a nucleic acid sequence encoding a variant peptide according to the first aspect. Preferably, the nucleic acid sequence comprises a nucleotide sequence substantially as set out in any one of SEQ ID No: 5-7. The nucleic acid sequence may be operably linked to a tissue-specific expression control sequence (such as a promoter), which drives expression of the nucleic acid sequence, wherein expression of the nucleic sequence results in the host organism displaying an altered phenotype.

The host organism may be a multicellular organism, which is preferably non-human. For example, the host organism may be a mouse, rat or Drosophila. The host may be used in studies of neurodegenerative disorders, preferably Alzheimer's disease.

As discussed in the Examples, the inventors have demonstrated that the variant Amyloid-beta (Aβ) peptide of the first aspect is non-toxic and does not aggregate into β-sheet structures, which would normally create amyloid plaques. The variant peptide therefore has significant utility in diagnosing Alzheimer's disease.

Thus, in a seventh aspect, there is provided the variant Amyloid-beta (Aβ) peptide of the first aspect, for use in diagnosing Alzheimer's disease.

Furthermore, the variant Aβ peptide of the invention can be used as a robust negative control for validating any effects of Aβ.

Hence, in a eighth aspect, there is provided use of the variant Amyloid-beta (Aβ) peptide of the first aspect, as a negative control in an Aβ study.

The inventors have developed a kit comprising the variant Amyloid-beta (Aβ) peptide for use in performing a wide range of Aβ studies or assays.

Thus, in a ninth aspect, there is provided an Amyloid-beta (Aβ) test kit comprising the variant Amyloid-beta (Aβ) peptide of the first aspect.

The kit preferably comprises a container in which the variant Aβ peptide is contained. The kit preferably comprises wild-type Aβ peptide, preferably in a container.

The wild-type Aβ (1-42) and variant Aβ 42 are prepared using the protocol described in the Examples in an identical way to ensure consistent and comparable starting peptides in disaggregated form, which can then be used in the subsequent assays in a detection kit.

Preferably, therefore, the kit comprises a solvent to disassemble any pre-aggregated peptide. The solvent is preferably in the container in which the or each peptide is contained. The solvent may be hexafluoroisopropanol. The solvent may be dimethylsulphoxide. Preferably, the kit comprises hexafluoroisopropanol and dimethylsulphoxide.

Preferably, the kit comprises a buffer, such as HEPES, phosphate buffer, or MOPS etc. Preferably, the kit comprises a desalting column.

Preferably, the kit is configured to be used in a variety of assays to explore the effects of the Alzheimer's Aβ by comparing wild-type Aβ with the variant Aβ.

Thus, in a tenth aspect, there is provided use of the variant Amyloid-beta (Aβ) peptide according to the first aspect, or the kit of the ninth aspect, in an assay selected from an aggregation assay; cell toxicity assay; animal assay, such as behavioural tests, molecular, cellular or tissue changes; cell uptake assay; membrane permeation assay; Aβ localisation assay using live cell imaging and immunofluorescence; immunogold electron microscopy; and molecular studies to compare and contrast the action and behaviour of the wild-type Aβ to the control variant Aβ.

The assay will provide valuable information about the specific effects of wild type Aβ for understanding its role in Alzheimer's disease. Any of these assays could include the addition of test compounds, but this is not necessary, as many of the assays will focus on finding targets and understanding the biochemical effects rather than drug discovery per se.

Thus, the variant Aβ peptide of the first aspect or the kit of the ninth aspect enables robust experiments to be performed for testing the efficacy of potential therapies that target Aβ toxicity or aggregation.

Hence, in a eleventh aspect, there is provided an assay for screening for a compound that modulates aggregation or toxicity of wild-type Aβ peptide, the assay comprising:—
(a) providing an assay system comprising wild-type Aβ peptide and the variant Aβ peptide of the first aspect;
(b) introducing a test compound into the assay system and determining the extent of aggregation and/or toxicity of the wild-type Aβ peptide;
(c) comparing the extent of aggregation and/or toxicity of the wild-type Aβ peptide
with that of the variant Aβ peptide of the first aspect,
   wherein an alteration in aggregation and/or toxicity of the wild-type Aβ peptide in the presence of the test compound compared to that of the variant Aβ peptide indicates that the test compound is a modulator of aggregation or toxicity of wild-type Aβ peptide.

The inventors have also designed a method of screening for useful therapeutic agents for preventing or treating Alzheimer's disease.

Hence, in a twelfth aspect, the invention provides a method of screening for a therapeutic agent useful in the prophylaxis or treatment of Alzheimer's disease, the method comprising:—
(a) introducing a test compound into an assay system comprising wild-type Aβ peptide; and
(b) determining aggregation and/or toxicity of the wild-type Aβ peptide,
   wherein an alteration in aggregation and/or toxicity of the wild-type Aβ peptide in the presence of the test compound compared to aggregation and/or toxicity of the variant Aβ peptide of the first aspect is an indication of the ability of the test compound to modulate Alzheimer's disease.

The assays and methods of the invention are preferably carried out in a kit.

Accordingly, in a thirteenth aspect, there is provided a kit for screening for a compound that modulates aggregation or toxicity of wild-type Aβ peptide, the kit comprising:—
(a) wild-type Aβ peptide; and
(b) the variant Aβ peptide of the first aspect,
   wherein the kit is configured to identify an alteration in aggregation and/or toxicity of the wild-type Aβ peptide in the presence of a test compound compared to that of the variant Aβ peptide, which alteration indicates that the test compound is a modulator of aggregation or toxicity of wild-type Aβ peptide.

Advantageously, and preferably, the variant Aβ peptide of the first aspect acts as a control, and provides the means for direct comparison of the effect of a potential therapeutic agent or compound aimed at intervening toxic effects of the wild-type Aβ peptide. In any experiment in which wild-type Aβ causes toxic effects, the variant control peptide would provide a direct comparison of a non-toxic form. Accordingly, introduction into the assay system of an agent or compound, which is active and therefore therapeutic, would be expected to return the experimental setup using wild-type Aβ peptide (i.e. its aggregation or toxicity) so that it is comparable with that of the variant control peptide. Such a test compound may therefore be used as a therapeutic agent useful in the prophylaxis or treatment of Alzheimer's disease. On the other hand, introduction into the assay system or kit of an agent or compound, which is not active or therapeutic, would result in greater aggregation/toxicity of wild-type Aβ peptide compared to that of the variant peptide.

The assay system used in step (a) in the assay or the method may be an in vitro, an in vivo or ex vivo system. The assay system may be a non-human animal model. For example, the animal may be a transgenic ape, monkey, mouse, rat, fish, ferret, sheep, dog, cat, worm or Drosophila.

The kit preferably comprises a container in which the wild-type Aβ peptide is contained. The kit preferably comprises a container in which the variant Aβ peptide is contained. Preferably, the kit comprises a solvent to disassemble any preaggregated peptide, for example hexafluoroisopropanol and/or dimethylsulphoxide. Preferably, the kit comprises a buffer, such as HEPES, phosphate buffer, or MOPS etc. Preferably, the kit comprises a desalting column.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No: 1-7, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, we mean the nucleotide hybridises to filter-bound DNA or RNA in 3x sodium chloride/sodium citrate (SSC) at approximately 45° C. followed by at least one wash in 0.2x SSC/0.1% SDS at approximately 20-65° C. Alternatively, a substantially similar polypeptide may differ by at least 1, but less than 5, 10, 20, 50 or 100 amino acids from the sequences shown in SEQ ID No: 1-4.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 is a graph produced using WALTZ and shows two peaks that indicate two amyloidogenic regions (residues 16-21 and residues 37-42) in the wildtype “Aβ(1-42)” peptide. The amino acids selected for substitution are highlighted in bold, i.e. F and G;

FIG. 2 shows the output from WALTZ for a variant peptide, “vAβ42”, in accordance with an embodiment of the invention, showing abolition of the amyloidogenic regions due to substitution of F with S, and of G with D;

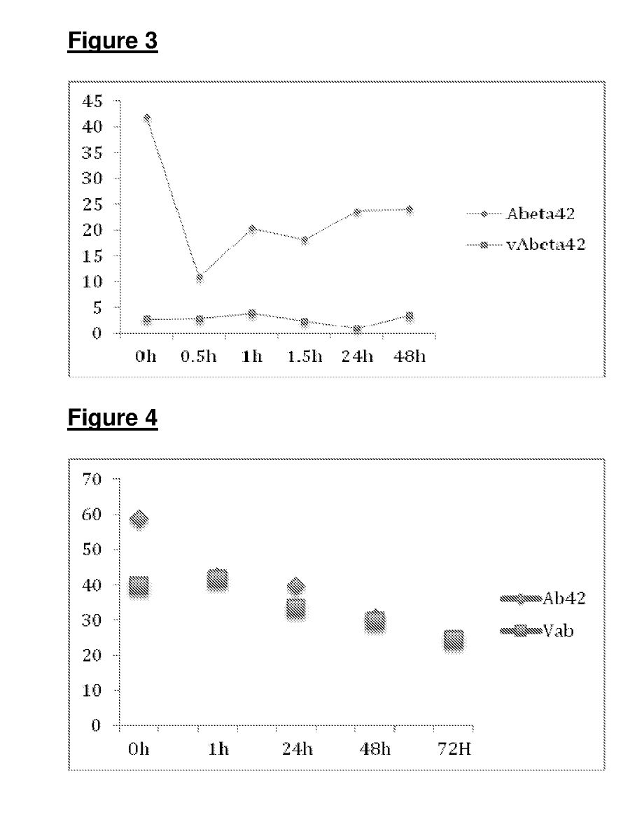

FIG. 3 shows a thioflavine T fluorescence fibril formation assay showing increasing fluorescence of wild type Aβ(1-42) with time, compared to no change in fluorescence of the variant, vAβ42;

FIG. 4 shows tyrosine fluorescence measured at 300 nm and reveals that both wild type Aβ (1-42) and vAβ42 undergo some conformational changes in the environment of the tyrosine 10 residue;

FIG. 5 shows CD spectra of wild type Aβ(1-42) with time showing that the wild type peptide forms β-sheet structures rapidly leading to amyloid plaques;

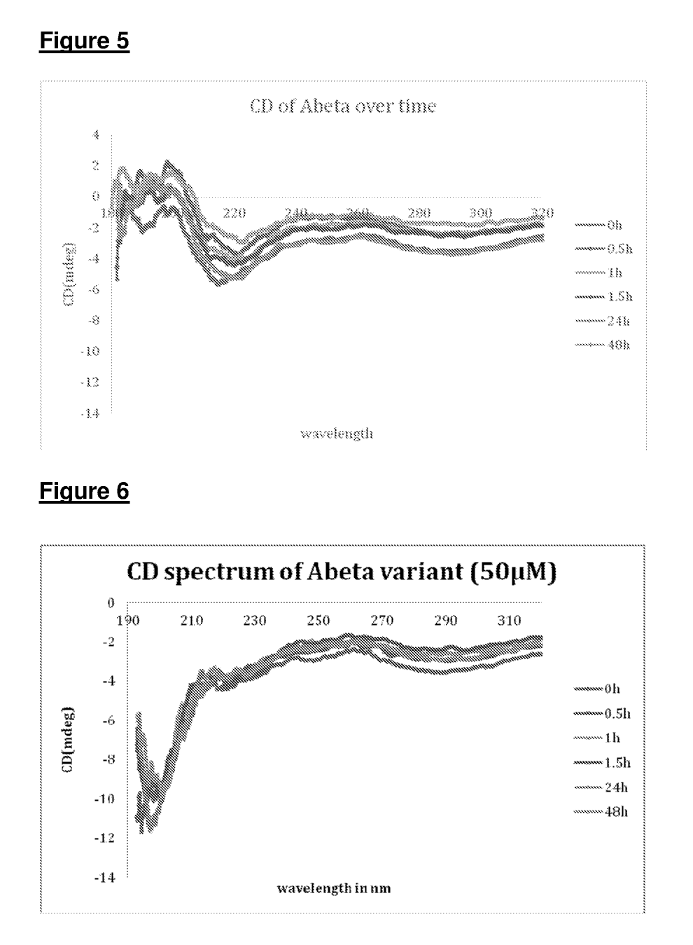

FIG. 6 shows CD spectra of variant vAβ42 with time showing that vAβ42 remains as a random coiled structure up to the final time point of 48 h, and that no β-sheet structures are formed;

FIG. 7 shows negative stain transmission electron microscopy. FIG. 7(a) shows fibrillar structures formed by wild type Aβ(1-42) after 48 hours compared to the amorphous structures formed by the variant vAβ42 in FIG. 7(b);

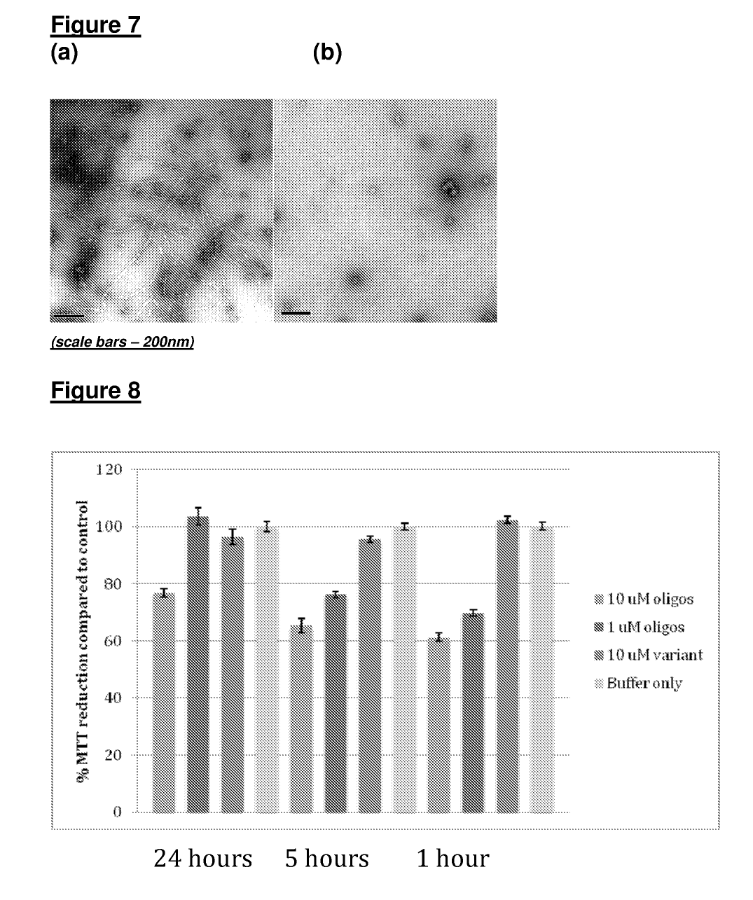

FIG. 8 shows MIT assays that measure the metabolic activity of SH-SY5Y cells and shows that oligomeric wild type Aβ(1-42) 1 and 10 μM have a significant effect on the cells after 24 hours, whilst variant vAβ42 is the same as buffer only;

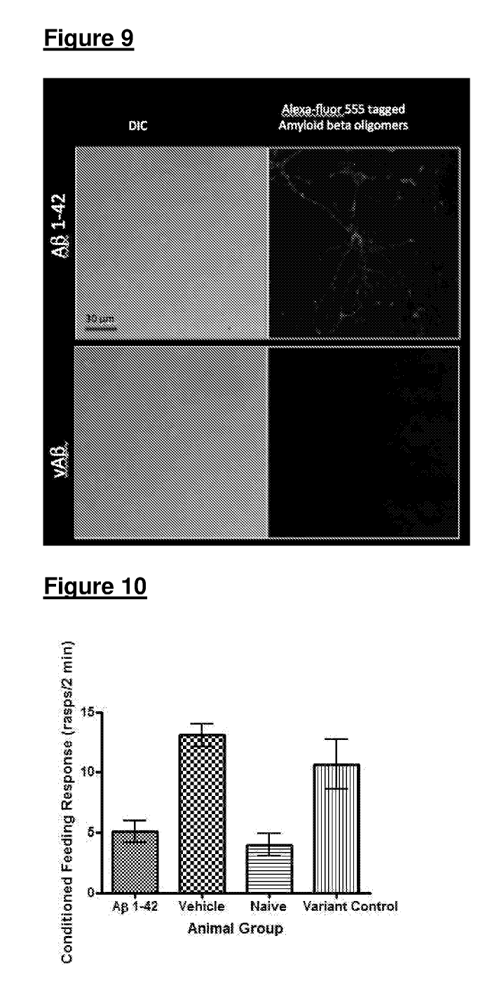

FIG. 9 shows that Aβ(1-42) enters neurons and is rapidly distributed through out processes and cell body whilst vAβ does not appear to enter neurons. AlexaFluor555 tagged Aβ(1-42) and vAβ were incubated with neurons for 24 hours and visualised using confocal microscopy. Figure shows differential interference contrast (DIC) compared to the confocal red channel showing the AlexFluor555 tag; and

FIG. 10 shows that Aβ(1-42) disrupts long term memory after 24 hour in vivo incubation, whilst vAβ does not. One-way ANOVA p<0.0001 Aβ(1-42) n=55, vehicle n=106, naïve n=65, variant control n=20 Snails were tested for rasp rate to amyl acetate, a measure of the feeding response to the CS. Means ± standard error mean (SEM) values are shown. Asterisks indicate behavioural responses that are significantly lower (***=p<0.0001, *=p<0.05) than those in the vehicle-treated group. One-way ANOVA p<0.0001 Tukey's multiple comparison: Aβ(1-42) v Vehicle: p<0.001; vehicle v naïve: p<0.001; variant control v naïve: p<0.05. All others p>0.05

### EXAMPLES

### Materials and Methods

Peptide Design

Sequence based design was performed using the WALTZ algorithm (2) to explore the effect of amino-acid substitutions on the predicted amyloidogenicity of the wildtype Aβ peptide. The graph produced using WALTZ shows two peaks that indicate the location of two amyloidogenic regions (residues 16-21 and residues 37-42) in the wildtype Aβ(1-42) peptide. Substitutions were introduced within the predicted amyloidogenic regions to examine the effect on the graphical output prediction. A number of variants were shown to reduce the predicted amyloidogenicity and two were selected based on literature studies as well as output from WALTZ.

Preparation and Systemic Application of A β and Variant Peptides

Wild-type Aβ (1-42) peptide was purchased from rPeptide (http://www.rpeptide.com). Synthetic variant Aβ peptide, “vAβ42”, was purchased from JPT (jpt.com). Both peptides were prepared in the same way using a preparation previously described which uses HFIP and DMSO to solubilize the peptides followed by complete removal of solvents (3, 4). Peptides were prepared in HEPES buffer (10 mM HEPES, 50 mM NaCl, 1.6 mM KCl, 2 mM MgCl₂, 3.5 mM CaCl₂), designed to mimic the culture media as previously described ^{8, 9}. Briefly, 0.2 mg Ab 1-42 (rPeptide) was solubilized in 200 μL HFIP (Sigma-Aldrich) to disaggregate the peptide. The solution was then vortexed on high for one minute and sonicated in a 50/60 Hz bath sonicator for five minutes. HFIP was dried completely using a low stream of nitrogen gas for five to ten minutes. Once completely dried, 200 μL dry DMSO (Sigma-Aldrich) was added, vortexed for one minute, and sonicated for one minute. Solutions were added to a Zeba buffer-exchange column equilibrated with HEPES buffer with 40 μL HEPES as a stacking buffer. The protein solution was kept on ice and the absorbance at 280 nm measured with a NanoDrop spectrophotometer using a molar absorption coefficient of 1490 M⁻¹ cm⁻¹. Solutions were immediately diluted to 50 μM with HEPES buffer and incubated for two hours, by which point oligomers are known to form in Aβ 1-42 preparations, before using in further experiments.

Thioflavine T Fluorescence

The sample was prepared with 3.121 μM of ThT in a 10 μM Aβ peptide and added to a 10 mm cuvette. An emission scan between a wavelength of 460 nm-600 nm was performed in a Varian Cary Eclipse Fluorescence Spectrophotometer. The sample compartment was set to 21° C., scan rate of 600 nm/min was used and 3 spectra were averaged for each measurement to improve accuracy.

Tyrosine Fluorescence

130 μl of 50 μM Aβ peptide was added to a 10 mm cuvette and an emission scan between wavelength 290 nm-500 nm was performed in a Varian Cary Eclipse Fluorescence Spectrophotometer. The sample compartment was set to 20° C., scan rate of 300 nm/min was used and 3 spectra were averaged for each measurement to improve accuracy.

Circular Dichroism

500 μl of 50 μM Aβ sample was placed into a 1 mm path length quartz cuvette (Hellma) and was scanned between 180 nm to 275 nm on a JASCO Spectropolarimeter J715.3 spectra were averaged for each measurement to improve accuracy and the samples were equilibrated at 20° C. using a water bath.

Transmission Electron Microscopy

TEM grids were prepared using Formvar/carbon film (Agar scientific) coated, 400 mesh copper grids. 4 μl of 50 μM Aβ was placed on the surface of the grid and allowed to be absorbed for 60 s and blotted dry. A 4 μl aliquot of miliQ-filtered water was then added to the grid and blotted dry after 60 s. Immediately after this the grid was negatively stained with 4 μl of 2% (w/v) uranyl acetate for 60 s and blotted dry. The uranyl acetate wash was repeated once more and the grid was left to air dry. All the TEM grids were examined using a Hitachi-7100 TEM at 100 kV and the images were acquired digitally with an axially mounted (2000×2000 pixel) Gatan Ultrascan 1000 CCD camera. Aliquots of Aβ peptide samples were taken at different time points to monitor the fibrillation state and morphology.

Cell Metabolism Assays

The Vybrant MTF [3-(4,5-dimethylthiazol-2-yl)-2,5-diphenyl-2H-tetrazolium bromide] cell-proliferation assay (Invitrogen) was used according to the manufacturer's protocol to assess the toxic effect of Aβ42 oligomers on undifferentiated SH-SY5Y cells. SH-SY5Y cells (2×10⁵ cells/well) were seeded on uncoated or collagen-I-coated glass coverslips in a 24-well plate 1 day prior to the assay. The cells were incubated 10 or 25 μmol.dm⁻³ oligomeric Aβ42 or variant Aβ for 1, 5 or 24 h at 37° C. At specific time points, 12 mmol.dm⁻³ MTT solution was added to the cells and further incubated for 2 hours at 37° C. The resulting insoluble dye was dissolved with 50 μL of DMSO and the fluorescence measured at 540 nm with a 620 nm reference filter. Untreated cells served as a reference and the value was set to 100% redox activity and compared to the treated cells, and then converted to percentage survival.

Immunofluorescence Comparison of Aβ 1-42 Compared to vAβ Internalisation

Aβ was tagged with AlexaFluor555 or 488 as previously described⁸. Briefly, the above protocol was followed up until the addition of DMSO. 10 μL of 113 nM Alexa fluor dye and 20 μL 1M sodium bicarbonate were added to Aâ₄₂ in DMSO. This was incubated for 15 minutes and the remaining stages of the protocol for Aâ preparation carried out. Alexa Fluor tagged Aβ was added to P0-P1 primary rat hippocampal neurons and incubated the desired length of time, after which the cells were washed once quickly with warmed EBS (external bath solution: 137 mM NaCl, 5 mM KCl, 3 mM CaCl₂, 1 mM CaCl₂, 10 mM D-Glucose, 5 mM HEPES) fixed in 2% paraformaldehyde for 15 minutes, washed three times for 5 minutes each with PBS and mounted in Prolong Gold (Life Technologies).

For samples that were additionally immunolabelled after incubation with Abeta, cells were first washed and fixed as above, then washed with wash buffer (25% Superblock (Thermo Scientific) diluted into PBS) and permeabilised with 0.3% Triton X-100 for 10 minutes. 50 mM Glycine was added for 5 minutes then the cells were washed and incubated for 30 minutes with Image-iT FX signal enhancer then blocked with Superblock (Thermo Scientific) for 30 minutes. Anti-Abeta oligomer antibody NU1 was added, followed by secondary antibody Anti-Mouse Alexa fluor 555 conjugate, each for 1 hour. Cells were then washed and mounted as described above.

Confocal Microscopy and Image Overlay

Samples were imaged on a Leica TCS SP8 using a 63x oil objective. Z stacks were taken with 0.5 um step size and images shown are maximal projections of the stack. Images were prepared using images prepared using FIJI software¹⁰.

Memory Test in Lymnaea Stagnalis

Pond snails, Lymnaea stagnalis, were bred at the University of Sussex and maintained in large holding tanks containing 18-22° C. copper-free water, at a 12:12 hour light-dark cycle. The animals were fed Tetra-Phyll (TETRA Werke) twice a week and lettuce three times a week. The peptides were administered to the animals directly after preparation. Using a 1 mL syringe with 30 gauge precision glide needles (Becton Dickinson), 100 μL of the Aβ1-42 or variant control peptide solution was injected into the haemolymph (˜1 mL in volume) of each snail. The estimated final concentration in the animal was 0.1 μM for Aβ1-42 and variant conrol. As there is no blood-brain barrier in Lymnaea (Sattelle and Lane, 1972), the injected peptides have direct access to the animal's central nervous system. For vehicle-injected control animals, 100 μL of normal saline was injected.

Using well-established methods (I. Kemenes et al., 2006), four-to six-month-old snails were removed from their home tanks and starved in new tanks for two days at the same temperature and light dark cycle as the home tanks. After the starvation period, the animals underwent single-trial food-reward classical conditioning (Alexander et al., 1984) in which the CS (amyl acetate: 0.004% final concentration) and the US (sucrose: 0.6% final concentration) were paired. Initially, each individual snail was left to acclimatise in a 14 cm diameter Petri dish with 90 mL of 18-22° C. copper-free water for ten minutes. After the acclimatisation period, 5 mL of amyl acetate was added to the dish and after thirty seconds, 5 mL of sucrose was added. The snails were then left in their Petri dishes for two minutes, and then removed to their starvation tanks. Both the vehicle-injected and Aβ-injected groups were trained. The naïve groups were not trained, but underwent the same starvation/feeding schedule and handling.

All animals were tested with the CS. Each individual snail was left to acclimatise in a 14 cm-diameter Petri dish with 90 mL of 18-22° C. copper-free water for ten minutes. After the acclimatisation period, 5 mL of 18-22° C. copper-free water was added to the dish. Rasps, the animals' feeding movements, were manually counted for two minutes to determine a baseline rasping rate (number of rasps per two minutes) for each individual. After two minutes, 5 mL amyl acetate was added to the dish. Rasping was tracked for two minutes. Rasping rates were determined by subtracting the individual animal's baseline rasp from the amyl acetate induced rasp.

Data that passed the D'Agostino and Pearson omnibus normality test were subjected to parametric tests (one-way analysis of single variance [ANOVA] with Tukey's multiple comparison, or t-tests) to establish significance (criterion, p<0.05). GraphPad Prism software was used for all analyses.

Results

Design of a Variant Control Peptide Based on Wild Type Aβ(1-42)

Referring to FIG. 1, there is shown the wildtype Aβ(1-42) peptide comprising 42 amino acids. All 19 remaining amino acid substitutions were introduced into the two amyloidogenic regions in Aβ42 identified from WALTZ identified as residues 16-21 and residues 37-42. The amino acid substitutions that reduced the peaks for amyloidogenic regions were then shortlisted (see Table 1).

TABLE 1
Shows substitution in to the two amyloidogenic
regions identified by WALTZ that result in
removal of the amyloidogenic propensity peak.

| | Sequence substituted |
|---|---|
| Substitution at F19 | |
| that removed | |
| Amyloidogenic | |
| region 1 KLVFFA | |
| F19P (Proline) | KLVPFA (SEQ ID NO. 8) |
| F19D (aspartic acid) | KLVDFA (SEQ ID NO. 9) |
| F19R (Arginine) | KLVRFA (SEQ ID NO. in) |
| F19C (Cysteine) | KLVCFA (SEQ ID NO. 11) |
| F19Q (Glutamine) | KLVQFA (SEQ ID NO. 12) |
| F19G (Glycine) | KLVGFA (SEQ ID NO. 13) |
| F19H (Histidine) | KLVHFA (SEQ ID NO. 14) |
| F19K (Lysine) | KLVKFA (SEQ ID NO. 15) |
| F19M (Methionine) | KLVMFA (SEQ ID NO. 16) |
| F19S (Serine) | KLVSFA (SEQ ID NO. 17) |
| Substitutions at G37 | |
| that removed | |
| Amyloidogenic | |
| region 2 GGVVIA | |
| G37R (Arginine) | RGVVIA (SEQ ID NO. 18) |
| G37D (Aspartic acid) | DGVVIA (SEQ ID NO. 19) |
| G37C (Cysteine) | CGVVIA (SEQ ID NO. 20) |
| G37E (Glutamic acid) | EGVVIA (SEQ ID NO. 21) |
| G37Q (Glutamine) | QGVVIA (SEQ ID NO. 22) |
| G37H (Histidine) | HGVVIA (SEQ ID NO. 23) |
| G37I (Isoleucine) | IGVVIA (SEQ ID NO. 24) |
| G37L (Leucine) | LGVVIA (SEQ ID NO. 25) |
| G37K (Lysine) | LGVVIA (SEQ ID NO. 26) |
| G37M (Methionine) | MGVVIA (SEQ ID NO. 27) |
| G37F (Phenylalanine) | FGVVIA (SEQ ID NO. 28) |
| G37P (Proline) | PGVVIA (SEQ ID NO. 29) |
| G375 (Serine) | SGVVIA (SEQ ID NO. 30) |
| G37T (Threonine) | TGVVIA (SEQ ID NO. 31) |
| G37W (Tryptophan) | WGVVIA (SEQ ID NO. 32) |
| G37Y (Tyrosine) | YGVVIA (SEQ ID NO. 33) |
| G37V (Valine) | VGVVIA (SEQ ID NO. 34) |

Out of all the shortlisted substitutions that worked, double substitution F19S, G37D was selected, as shown in FIG. 2. Substituting phenylalanine at position 19 (F19) with Serine (S) and Glycine at position 37 (G37) with Aspartic acid (D) indicates removal of the amyloidogenic regions in the variant Aβ42 peptide (FIG. 2). Therefore, it can be hypothesised that substituting phenylalanine at position 19 that is tightly packed at a hydrophobic region with a smaller and less hydrophobic amino acid-serine and glycine at position 37 which is a small non-polar, neutral amino acid with a negatively charged and acidic aspartic acid has some affect in disrupting the intermolecular contacts and thus prevent variant vAβ42 from aggregating into amyloid plaques.

Referring to FIG. 3, Thioflavine T fluorescence was used to monitor amyloid assembly with time and showed that whilst wild type Aβ(1-42) showed an increasing fluorescent signal with time, there was no change in fluorescence at 483 nm for the variant vAβ42 suggesting that this peptide does not self-assemble.

Tyrosine fluorescence has been used previously to monitor the change in fluorescence as the Aβ peptide assembles and changes the environment of the tyrosine residue at position 10 (5). Referring to FIG. 4, there is shown tyrosine fluorescence measured at 300 nm and reveals that both wild type Aβ(1-42) and variant vAβ42 undergo conformational changes in the environment of the tyrosine 10 residue.

CD is used to monitor the conformational change from random coil to β-sheet structure that accompanies amyloid assembly. CD spectra confirm that whilst wild type rapidly forms β-sheet structures, the variant vAβ42 remains random coil conformation for the duration of the experiment, as shown in FIGS. 5 and 6. As such, the vAβ42 is not forming β-sheets which would create amyloid plaques in vivo.

Electron microscopy was used to examine the morphology of the structures over time. As shown in FIG. 7, after 48 hours, wild type Aβ(1-42) had formed fibrils as expected, whilst the variant vAβ42 forms small spherical structures that appear to be variable and amorphous after 48 hour incubation at 50 μM.

Wild type Aβ(1-42) has been shown to have a toxic effect on cultured neuroblastoma cells and neurons (6,7). In order to investigate the effect of the variant vAβ42 on cells and to compare to wild type Aβ(1-42), an MTT assay was conducted to assess the effect on metabolic activity of SH-SY5Y cells. As shown in FIG. 8, the results revealed that oligomeric wild type Aβ(1-42) decreases the metabolic activity of the neuroblastoma cells, whilst the variant vAβ has no effect and is comparable to vehicle buffer only.

Immunofluorescence Comparison of Wild Type AP Compared to Variant vAβ Internalisation

Tagged wild type Aβ(1-42) and variant vAβ42 were added to neuronal cultures and then visualised using a confocal microscope at time points following addition of 24 hours, as shown in FIG. 9. Clear differences in the pattern of uptake were observed between Aβ(1-42) and vAβ42. In particular, Aβ(1-42) appears to enter the cell body and to associate with the processes of the neurons, whilst vAβ42 is not observed and does not appear to enter the neurons.

Memory Test in Lymnaea Stagnalis

Aβ(1-42) and vAβ2 were administered to Lymnaea Stagnalis in a conditioned response memory test as previously described¹¹. In this test, FIG. 10 shows the reduction in rasp rate following Aβ(1-42) compared to the vehicle (buffer only control). Variant vAβ42 is denoted by “control peptide” and shows a similar rasp rate to the vehicle control showing that vAβ42 does not have the ability to alter the memory in the snails.

Peptide Preparation Kit

The inventors have developed a peptide preparation kit which includes the variant vAβ42 peptide as a control, and which can then be used in a variety of assays to explore the effects of the Alzheimer's Aβ. These assays could be wide ranging, including but not limited to:
a) aggregation assays;
b) cell toxicity assays; and
c) animal tests (behavioural tests, molecular, cellular or tissue changes).

The kit includes:—
(i) a vial containing wild-type Aβ(1-42);
(ii) a vial containing variant vAβ42 (i.e. the peptide of the invention);
(iii) solvent (Hexafluoroisopropanol);
(iv) solvent (Dimethylsulphoxide, dry);
(v) buffer (HEPES, PB etc); and
(vi) desalting column (Invitrogen).

The wild-type Aβ (1-42) and variant Aβ42 are prepared using the kit in an identical way to ensure consistent starting peptides in disaggregated form, which can then be used in the subsequent assays in a detection kit (described below). The solvents are provided to ensure that the peptides are disaggregated.

Aggregation/Toxicity Detection Kit

The two peptides are used in assays, including cell toxicity, cell uptake, membrane permeation, Aβ localisation using live cell imaging and immunofluorescence, immunogold electron microscopy, animal behaviour, molecular studies etc. to compare and contrast the action and behaviour of the wild-type Aβ to the control variant Aβ. This will provide valuable information about the specific effects of wildtype Aβ for understanding its role in Alzheimer's disease. Any of these assays could include the addition of test compounds, but this is not necessary, as many of the assays will focus on finding targets and understanding the biochemical effects rather than drug discovery per se.

In one embodiment, a test compound is added to the kit following preparation of the two peptides under the protocol contained within the preparation kit. A known amount of a test compound is introduced into the assay (cell toxicity etc), and the amount of aggregation and/or toxicity as detected and quantified, and compared. As discussed above, the variant Amyloid-beta (Aβ) peptide exhibits reduced propensity to aggregate compared to the wild type peptide, and so is used as a negative control against which aggregation of the wild type can be measured. An alteration in aggregation and/or toxicity of the wild-type Aβ peptide in the presence of the test compound compared to that of the variant Aβ peptide indicates that the test compound is a modulator of aggregation or toxicity of wild-type Aβ peptide. The kit can be used to screen a therapeutic agent useful in the prophylaxis or treatment of Alzheimer's disease.

### References

1. Sipe, J. D. (1992) Amyloidosis. Ann Rev Biochem 61, 947-975

2. Maurer-Stroh, S., Debulpaep, M., Kuemmerer, N., de la Paz, M. L., Martins, I. C., Reumers, J., Morris, K. L., Copland, A., Serpell, L., Serrano, L., Schymkowitz, J. W., and Rousseau, F. (2010) Exploring the sequence determinants of amyloid structure using position-specific scoring matrices. Nat Methods 7, 237-242

3. Broersen, K., Jonckheere, W., Rozenski, J., Vandersteen, A., Pauwels, K., Pastore, A., Rousseau, F., and Schymkowitz, J. (2011) A standardized and biocompatible preparation of aggregate-free amyloid beta peptide for biophysical and biological studies of Alzheimer's disease. Protein engineering, design & selection: PEDS 24, 743-750

4. Williams, T. L., Johnson, B. R., Urbane, B., Jenkins, A. T., Connell, S. D., and Serpell, L. C. (2011) Abeta42 oligomers, but not fibrils, simultaneously bind to and cause damage to ganglioside-containing lipid membranes. The Biochemical journal 439, 67-77

5. Al-Hilaly, Y. K., Williams, T. L., Stewart-Parker, M., Ford, L., Skaria, E., Cole, M., Bucher, W. G., Morris, K. L., Sada, A. A., Thorpe, J. R., and Serpell, L. C. (2013) A central role for dityrosine crosslinking of Amyloid-beta in Alzheimer's disease. Acta neuropathologica communications 1, 83

6. Soura, V., Stewart-Parker, M., Williams, T. L., Ratnayaka, A., Atherton, J., Gorringe, K., Tuffin, J., Darwent, E., Rambaran, R., Klein, W., Lacor, P., Staras, K., Thorpe, J., and Serpell, L. C. (2012) Visualization of co-localization in Abeta42-administered neuroblastoma cells reveals lysosome damage and autophagosome accumulation related to cell death. The Biochemical journal 441, 579-590

7. Irvine, G. B., El-Agnaf, O. M., Shankar, G. M., and Walsh, D. M. (2008) Protein aggregation in the brain: the molecular basis for Alzheimer's and Parkinson's diseases. Mol Med 14, 451-464

8. Soura, V. et al. Visualization of co-localization in Abeta42-administered neuroblastoma cells reveals lysosome damage and autophagosome accumulation related to cell death. The Biochemical journal 441, 579-590, doi:10.1042/BJ20110749 (2012).

9. Broersen, K. et al. A standardized and biocompatible preparation of aggregate-free amyloid beta peptide for biophysical and biological studies of Alzheimer's disease. Protein engineering, design & selection: PEDS 24, 743-750, doi:10.1093/protein/gzr020 (2011).

10. Schindelin, J. et al. Fiji: an open-source platform for biological-image analysis. Nature methods 9, 676-682, doi:10.1038/nmeth.2019 (2012).

11. Ford, L. et al. Effects of Abeta exposure on long-term associative memory and its neuronal mechanisms in a defined neuronal network. Scientific reports 5, 10614, doi:10.1038/srep10614 (2015).

1-33. (canceled)

34. A variant Amyloid-beta (Aβ) peptide comprising a modified amino acid sequence of a wild-type Aβ peptide, wherein the modified amino acid peptide exhibits reduced propensity to aggregate compared to the wild type peptide.

35. The variant Aβ peptide according to claim 34, wherein the variant Amyloid-beta (Aβ) peptide comprises one or more modification in amino acids 16-21 or 37-42 of SEQ ID No: 1.

36. The variant Aβ peptide according to claim 34, wherein the variant Amyloid-beta (Aβ) peptide comprises at least two modifications in amino acids 16-21 or 37-42 of SEQ ID No: 1.

37. The variant Aβ peptide according to claim 34, wherein the variant Amyloid-beta (Aβ) peptide comprises at least one modification in amino acids 16-21 of SEQ ID No: 1 and at least one modification in amino acids 37-42 of SEQ ID No: 1.

38. The variant Aβ peptide according to claim 34, wherein the variant Amyloid-beta (Aβ) peptide is formed by modification of amino acid residue F19 or G37 of SEQ ID No: 1.

39. The variant Aβ peptide according to claim 34, wherein the variant Amyloid-beta (Aβ) peptide is formed by modification of amino acid residue F19 and G37 of SEQ ID No: 1.

40. The variant Aβ peptide according to claim 35, wherein the modification at amino acid residue F19 comprises a substitution with a serine.

41. The variant Aβ peptide according to claim 34, wherein the variant Amyloid-beta (Aβ) peptide comprises an amino acid sequence substantially as set out in SEQ ID No: 2.

42. The variant Aβ peptide according to claim 35, wherein the modification at amino acid residue G37 comprises a substitution with an aspartic acid.

43. The variant Aβ peptide according to claim 34, wherein the variant Amyloid-beta (Aβ) peptide comprises an amino acid sequence substantially as set out in SEQ ID No: 3.

44. The variant Aβ peptide according to claim 34, wherein the variant Amyloid-beta (Aβ) peptide comprises a F19S substitution or a G37D substitution.

45. The variant Aβ peptide according to claim 34, wherein the variant Amyloid-beta (Aβ) peptide comprises an F19S substitution and a G37D substitution.

46. The variant Aβ peptide according to claim 34, wherein the variant Amyloid-beta (Aβ) peptide comprises an amino acid sequence substantially as set out in SEQ ID No: 4.

47. An isolated nucleic acid molecule encoding the variant Amyloid-beta (Aβ) peptide according to claim 34.

48. The isolated nucleic acid molecule according to claim 47, wherein the isolated nucleic acid molecule comprises a nucleotide sequence substantially as set out in any one of SEQ ID No: 5-7, or functional variant thereof.

49. An Amyloid-beta (Aβ) test kit comprising the variant Amyloid-beta (Aβ) peptide according to claim 34.

50. The kit according to claim 49, wherein the kit comprises wild-type Aβ peptide.

51. The kit according to claim 49, wherein the kit comprises a solvent to disassemble any pre-aggregated peptide, and optionally a buffer and/or a desalting column.

52. The kit according to claim 51, wherein the solvent is hexafluoroisopropanol and/or dimethylsulphoxide.
